(19) United States
(12) Patent Application Publication
(10) Pub. No.: US 2024/0277384 A1
(43) Pub. Date: Aug. 22, 2024

(54) INTERSPINOUS SPACER WITH ACTUATOR LOCKING ARRANGEMENTS AND METHODS AND SYSTEMS

(71) Applicant: Boston Scientific Neuromodulation Corporation, Valencia, CA (US)

(72) Inventor: Zdzislaw Bernard Malinowski, Castaic, CA (US)

(21) Appl. No.: 18/432,562

(22) Filed: Feb. 5, 2024

Related U.S. Application Data
(60) Provisional application No. 63/447,147, filed on Feb. 21, 2023.

(51) Int. Cl. A61B 17/70; A61B 17/00

(52) U.S. Cl. CPC A61B 17/7047; A61B 17/7065; A61B 2017/00367

(57) ABSTRACT

An interspinous spacer includes an actuator screw having a proximal end and a shaped cavity in the proximal end, where the shaped cavity is configured for receiving a bit of a driver tool having a complementary shape for rotating the actuator screw using the driver tool; a wedge coupled to the actuator screw, wherein, as the actuator screw is rotated using the driver tool, the actuator screw and the wedge move longitudinally relative to the body; and a first arm and a second arm, wherein the first and second arms are rotatably coupled to the distal portion of the body and configured for rotating in response to longitudinal movement of the wedge. The interspinous spacer may include locking inserts or a locking ring to resist rotation of the actuator screw absent the driver tool.

### CROSS-REFERENCE TO RELATED APPLICATIONS

This application claims the benefit under 35 U.S.C. § 119(e) of U.S. Provisional Patent Application Ser. No. 63/447,147, filed Feb. 21, 2023, which is incorporated herein by reference.

### FIELD

The present invention is directed to the area of interspinous spacers for deployment between adjacent spinous processes. The present invention is also directed to systems and methods for utilizing the interspinous spacer.

### BACKGROUND

With spinal stenosis, the spinal canal narrows and pinches the spinal cord and nerves, causing pain in the back and legs. Typically, with age, a person's ligaments may thicken, intervertebral discs may deteriorate, or facet joints may break down. The conditions can contribute to the narrowing of the spinal canal. Injury, heredity, arthritis, changes in blood flow, and other causes may also contribute to spinal stenosis.

Various treatments of the spine have been proposed or used including medications, surgical techniques, and implantable devices that alleviate and reduce pain associated with the back. In one surgical technique. a spacer is implanted between adjacent spinous processes of a patient's spine. The implanted spacer opens the spinal canal. maintains the desired distance between vertebral body segments, and, as a result, avoids or reduces impingement of nerves and relieves pain. For suitable candidates, an implantable interspinous spacer may provide significant benefits in terms of pain relief.

### BRIEF SUMMARY

One aspect is an interspinous spacer that includes a body having a distal portion, a proximal portion, a proximal surface, and a channel extending longitudinally from the proximal surface toward the distal portion; an actuator screw including a proximal end and a shaped cavity in the proximal end, wherein the shaped cavity is configured for receiving a bit of a driver tool having a complementary shape for rotating the actuator screw using the driver tool, wherein at least a portion of the actuator screw and the channel of the body include complementary threading, wherein the actuator screw is at least partially disposed in, and engages, the channel of the body; a wedge coupled to the actuator screw, wherein, as the actuator screw is rotated using the driver tool, the actuator screw and the wedge move longitudinally relative to the body; and a first arm and a second arm, wherein the first and second arms are rotatably coupled to the distal portion of the body and configured for rotating in response to longitudinal movement of the wedge, wherein the actuator screw, wedge, first arm, and second arm are configured, upon rotation of the actuator screw in a first direction, to rotate the first and second arms from an implantation position, in which the first and second arms extend longitudinally from the distal portion of the body, to a deployed position, in which the first and second arms are rotated from the implantation position.

In at least some aspects, in the deployed position, the first and second arms extend away from the body in a lateral direction. In at least some aspects, in the deployed position, the first and second arms are rotated from the implantation position by at least 75 degrees.

In at least some aspects, the portion of the actuator screw including the threading is a threaded shaft and the actuator screw further includes a disc and a cylinder coupling the disc to the threaded shaft, wherein the cylinder has an outer diameter that is smaller than outer diameters of the disc and the threaded shaft. In at least some aspects, the wedge defines an engagement slot for receiving the disc of the actuator screw and includes two overhang portions that extend over the engagement slot are separated by a distance that is greater than the outer diameter of the cylinder and less than the outer diameters of the disc and the threaded shaft. In at least some aspects the disc includes a plurality of teeth arranged around a perimeter of the disc. In at least some aspects, the interspinous spacer further includes at least one locking inset positioned on the wedge for engagement by the disc of the actuator screw, each of the at least one locking inset including at least one tooth for interaction with the teeth of the disc to limit rotation of the actuator screw absent the driver tool. In at least some aspects, the interspinous spacer further includes a locking ring configured for engagement with the actuator screw to limit rotation of the actuator screw absent the driver tool, wherein the body includes a plurality of fingers extending above the proximal surface to retain the locking ring on the proximal surface of the body.

In at least some aspects, the interspinous spacer further includes two pins, wherein the body includes two sets of opposing pin holes in the distal portion and the first and second arm include at least one pin hole, wherein each of the pins couples either the first or second arm to the body and extends through one of the sets of opposing pin holes of the body and the pin hole of the corresponding one of the first arm or second arm. In at least some aspects, the body includes opposing undercut notches configured for receiving a clamp of a spacer insertion instrument.

Another aspect is an interspinous spacer that includes a body having a distal portion, a proximal portion, a proximal surface, and a channel extending longitudinally from the proximal surface toward the distal portion; an actuator screw including a threaded shaft and a disc coupled to the threaded shaft, wherein the disc includes a plurality of teeth arranged around a perimeter of the disc and the channel of the body includes threading complementary to the threaded shaft, wherein the actuator screw is at least partially disposed in, and engages, the channel of the body; a wedge coupled to the actuator screw, wherein, as the actuator screw is rotated, the actuator screw and the wedge move longitudinally relative to the body; at least one locking inset positioned on the wedge for engagement by the disc of the actuator screw, each of the at least one locking inset including at least one tooth for interaction with the teeth of the disc to limit rotation of the actuator screw; and a first arm and a second arm, wherein the first and second arms are rotatably coupled to the distal portion of the body and configured for rotating in response to longitudinal movement of the wedge, wherein the actuator screw, wedge, first arm, and second arm are configured, upon rotation of the actuator screw in a first direction, to rotate the first and second arms from an implantation position, in which the first and second arms extend longitudinally from the distal portion of the body, to a deployed position, in which the first and second arms are rotated from the implantation position.

In at least some aspects, in the deployed position, the first and second arms extend away from the body in a lateral direction. In at least some aspects, the actuator screw further includes a cylinder coupling the disc to the threaded shaft, wherein the cylinder has an outer diameter that is smaller than outer diameters of the disc and the threaded shaft, wherein the wedge defines an engagement slot for receiving the disc of the actuator screw and includes two overhang portions that extend over the engagement slot are separated by a distance that is greater than the outer diameter of the cylinder and less than the outer diameters of the disc and the threaded shaft. In at least some aspects, the at least one locking insert is positioned within the engagement slot of the wedge.

A further aspect is an interspinous spacer that includes a body having a distal portion, a proximal portion, a proximal surface, and a channel extending longitudinally from the proximal surface toward the distal portion; an actuator screw including a threaded shaft and a disc coupled to the threaded shaft, wherein the channel of the body includes threading complementary to the threaded shaft and the actuator screw is at least partially disposed in, and engages, the channel of the body; a wedge coupled to the actuator screw, wherein, as the actuator screw is rotated, the actuator screw and the wedge move longitudinally relative to the body; a locking ring configured for engagement with the actuator screw to limit rotation of the actuator screw, wherein the body includes a plurality of fingers extending above the proximal surface to retain the locking ring on the proximal surface of the body; and a first arm and a second arm, wherein the first and second arms are rotatably coupled to the distal portion of the body and configured for rotating in response to longitudinal movement of the wedge, wherein the actuator screw, wedge, first arm, and second arm are configured, upon rotation of the actuator screw in a first direction, to rotate the first and second arms from an implantation position, in which the first and second arms extend longitudinally from the distal portion of the body, to a deployed position, in which the first and second arms are rotated from the implantation position.

In at least some aspects, the actuator screw further includes a cylinder coupling the disc to the threaded shaft, wherein the cylinder has an outer diameter that is smaller than outer diameters of the disc and the threaded shaft, wherein the wedge defines an engagement slot for receiving the disc of the actuator screw and includes two overhang portions that extend over the engagement slot are separated by a distance that is greater than the outer diameter of the cylinder and less than the outer diameters of the disc and the threaded shaft. In at least some aspects, the interspinous spacer further includes two pins, wherein the body includes two sets of opposing pin holes in the distal portion and the first and second arm include at least one pin hole, wherein each of the pins couples either the first or second arm to the body and extends through one of the sets of opposing pin holes of the body and the pin hole of the corresponding one of the first arm or second arm.

A further aspect is a kit that includes any of the interspinous spacers described above; a spacer insertion instrument configured to releasably grip the interspinous spacer for implantation into a patient; and a driver tool including a spacer engaging bit configured to engage the actuator screw of the interspinous spacer and rotate the actuator screw by rotation of the driver tool.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified. For a better understanding of the present invention, reference will be made to the following Detailed Description, which is to be read in association with the accompanying drawings, wherein:

FIG. 1A is a schematic perspective view of one embodiment of an interspinous spacer in a deployed position;

FIG. 1B is a schematic perspective exploded view of the interspinous spacer of FIG. 1A;

FIG. 2A is a schematic side view of the interspinous spacer of FIG. 1A in an undeployed position;

FIG. 2B is a schematic side view of the interspinous spacer of FIG. 1A in the deployed position;

FIG. 3A is schematic perspective side view of a body of the interspinous spacer of FIG. 1A;

FIG. 3B is schematic perspective bottom view of a body of the interspinous spacer of FIG. 1A;

FIG. 4 is schematic perspective side view of an actuator screw, wedge, and locking inserts of the interspinous spacer of FIG. 1A;

FIG. 5A is a schematic perspective view of another embodiment of an interspinous spacer in a deployed position;

FIG. 5B is a schematic perspective exploded view of the interspinous spacer of FIG. 5A;

FIG. 6A is a schematic side view of the interspinous spacer of FIG. 5A in an undeployed position;

FIG. 6B is a schematic side view of the interspinous spacer of FIG. 5A in the deployed position;

FIG. 7A is schematic perspective side view of a body of the interspinous spacer of FIG. 5A;

FIG. 7B is schematic perspective bottom view of a body of the interspinous spacer of FIG. 5A;

FIG. 8 is schematic perspective side view of an actuator screw and wedge of the interspinous spacer of FIG. 5A;

FIG. 9 is a perspective view of one embodiment of a spacer insertion instrument; and

FIG. 10 is a perspective view of one embodiment of a driver tool.

### DETAILED DESCRIPTION

The present invention is directed to the area of interspinous spacers for deployment between adjacent spinous processes. The present invention is also directed to systems and methods for utilizing the interspinous spacer.

Examples of interspinous spacers are found in U.S. Pat. Nos. 8,123,782; 8,128,662; 8,273,108; 8,277,488; 8,292,922; 8,425,559; 8,613,747; 8,864,828; 9,119,680; 9,155,572; 9,161,783; 9,393,055; 9,532,812; 9,572,603; 9,861,398; 9,956,011; 10,080,587; 10,166,047; 10,610,267; 10,653,456; 10,835,295; 10,835,297; 11,013,539; and 11,229,461, all of which are incorporated herein by reference. Unless indicated otherwise, the features and methods described in these references can be applied to the interspinous spacers described herein.

In at least some of the spacers in the references cited above, a spindle arrangement is provided for engagement by a driver tool and rotation using the driver tool to cause the arms of the spacer to rotate for engagement with the vertebrae (for example the spinous processes of the adjacent vertebrae). The spindle arrangement is welded to the body of the interspinous spacer.

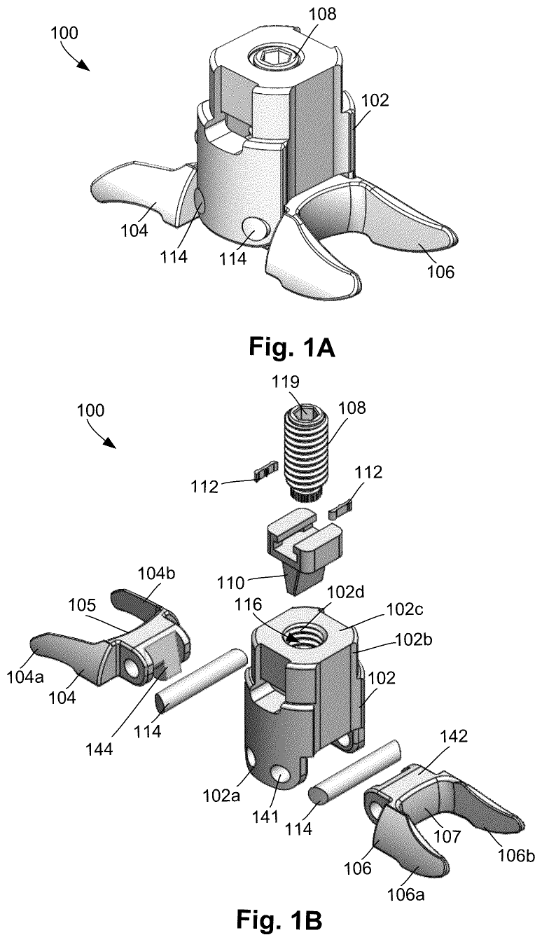

In contrast, a spacer can utilize an actuator screw instead of a spindle arrangement. FIGS. 1A (perspective view) and 1B (exploded view) illustrate one embodiment of an interspinous spacer 100 that includes a body 102, a first (or superior) arm 104, a second (or inferior) arm 106, an actuator screw 108, a wedge 110, two locking inserts 112, and two pins 114. There is no weld between the body 102 and the actuator screw 108.

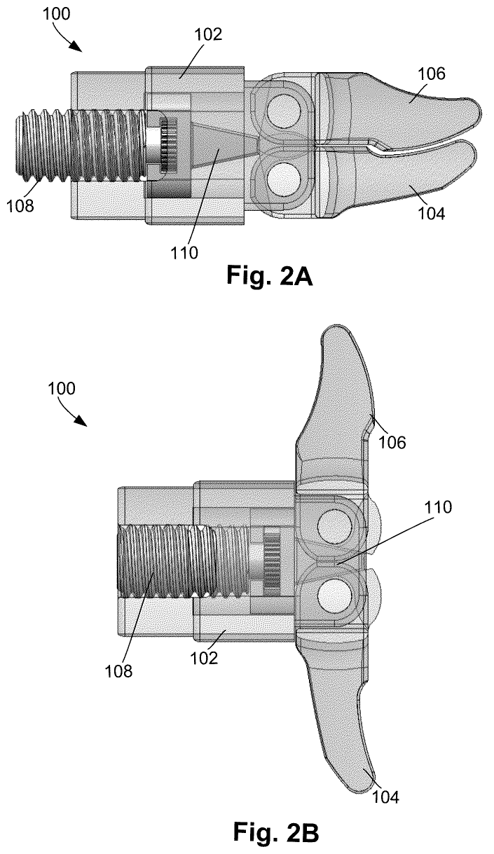

In FIGS. 1A and 2B, the first and second arms 104, 106 of the spacer 100 are in the deployed position with the arms 104, 106 extending away from the body 102 for receiving portions of the adjacent vertebrae, such as, for example, the spinous processes or other portions of the vertebrae. In the implantation position (e.g., undeployed position) illustrated in FIG. 2A, the first and second arms 104, 106 of the spacer 100 extend distally from the distal portion 102a of the body 102.

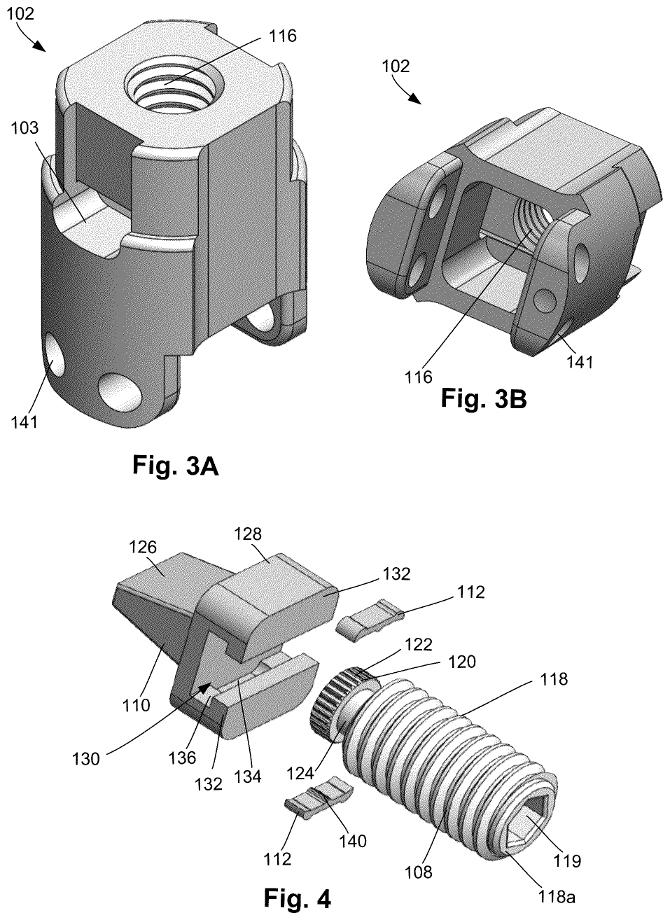

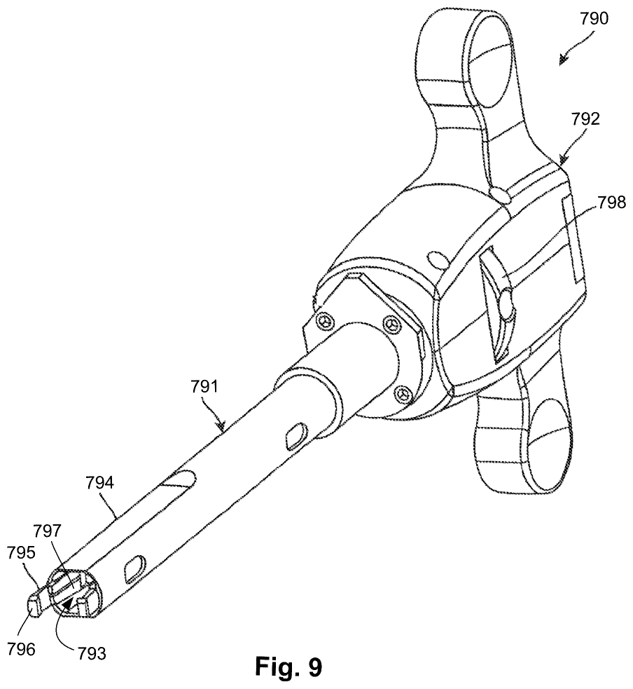

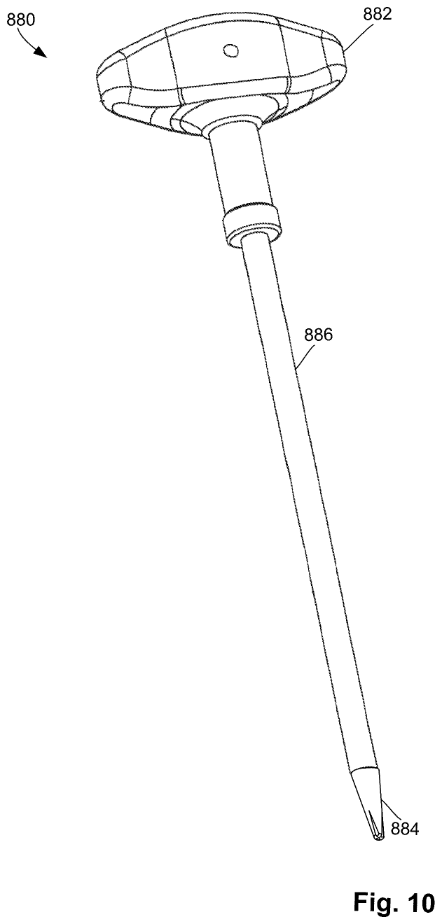

The body 102 includes a distal portion 102a, a proximal portion 102b, a proximal surface 102c, and an opening 102d in the proximal surface for the actuator screw 108. The body 102 defines a channel 116 that extends distally from the opening 102d through at least a portion of the body 102, as illustrated in FIGS. 3A and 3B. The channel 116 of spacer 100 is threaded. In at least some embodiments, the body 102 includes undercut notches 103 formed on opposite sides of the proximal portion 102b of the body. In at least some embodiments, the notches 103 are configured for attachment of clamps 795 of a spacer insertion instrument 790 (FIG. 9).

The actuator screw 108 includes a threaded shaft 118, a disc 120 with teeth 122 arranged around the perimeter of the disc, and a cylinder 124 coupling the threaded shaft to the disc, as illustrated in FIG. 4. The cylinder 124 has an outer diameter that is smaller than the outer diameters of the threaded shaft 118 and the disc 120. The actuator screw 108 can be made from a single piece of material or may contain two or more components that are attached together. The proximal end 118a of the threaded shaft 118 of the actuator screw 108 has a shaped cavity 119 to receive a driver tool 880 (FIG. 10) with a complementary-shaped engaging bit 884. Engagement of the actuator screw 108 by the driver tool allows a user to rotate the actuator screw to deploy (and, in at least some embodiments, retract) the first and second arms 104, 106. The threads and the size of the channel 116 of the body 102 and the threaded shaft 118 of the actuator screw 108 are complementary so that the actuator screw 108 fits within the channel 116 and moves distally or proximally, along a path defined by the threads, as the actuator screw 108 is rotated.

The wedge 110 includes a wedge portion 126 that interacts with the first and second arms 104, 106 and an actuator receiver 128 attached to the wedge portion for coupling to the actuator screw 108. The actuator receiver 128 defines an engagement slot 130 for receiving the disc 120 of the actuator screw 108 and the locking inserts 112. The actuator receiver 128 includes two overhang portions 132 that extend over the engagement slot 130 and are separated by a distance that is greater than the outer diameter of the cylinder 124 of the actuator screw 108, but less than the outer diameter of the disc 120 and the threaded shaft 118. The overhang portions 132 facilitates retention of the disc 120 of the actuator screw 108 in the engagement slot 130 while allowing rotation of the actuator screw.

The locking insets 112 fit within indents 134 in the interior walls 136 of the actuator receiver 128 of the wedge 110 so that the locking insets 112 are exposed within the engagement slot 130. Each locking inset 112 includes a body 138 and at least one tooth 140 extending from the body into the engagement slot 130 when the locking inset is disposed within the actuator receiver 128 of the wedge 110. The at least one tooth 140 of the locking inset 112 is arranged to engage the teeth 122 of the disc 120 of the actuator screw 108 when the disc is disposed within the engagement slot. The at least one tooth 140 of the locking insets 112 and the teeth 122 of the disc 120 of the actuator screw 108 are arranged to resist or prevent rotation of the actuator screw except by use of a driver tool 880 (FIG. 10) that engages the actuator screw 108. In at least some embodiments, the shape and size of the at least one tooth 140 of the locking insets 112 and the teeth 122 of the disc 120 of the actuator screw 108 are selected to resist or prevent rotation of the actuator screw when the first and second arms 104, 106 are deployed and force is applied to the first and second arms 104, 106 as the patient moves or bends. In at least some embodiments, the shape and size of the at least one tooth 140 of the locking insets 112 and the teeth 122 of the disc 120 of the actuator screw 108 are selected to generate a clicking sound as the actuator screw 108 is rotated using the driver tool 880 (FIG. 10).

The first and second arms 104, 106 are coupled to the distal portion 102a of the body 102 and coupled to the actuator 108 for rotation of the arms as described below. The first arm 104 includes two extensions 104a, 104b coupled by a bridge 105 from which an attachment portion 142 extends. The second arm 106 includes two extensions 106a, 106b coupled by a bridge 107 from which the attachment portion 142 extends. When the first and second arms 104, 106 are deployed, as illustrated in FIG. 1A, the pairs of extensions 104a, 104b, 106a, 106b extend away from the body 102 of the spacer 100 with the extensions of each pair disposed on opposing sides of one of the adjacent vertebrae (for example, the spinous process of the adjacent vertebra).

In at least some embodiments, during deployment, the first and second arms 104, 106 rotate through an arc of approximately 90 degrees (but is at least 30, 45, 60, 75, 80, 85, or 90 degrees) with respect to the body 102 to the deployed position in which the extensions 104a, 104b, 106a, 106b of the first and second arms are approximately perpendicular to the longitudinal axis of the body 102 as shown in FIG. 1A. In at least some embodiments, the first and second arms 104, 106 have a U-shaped projection in a plane perpendicular to the longitudinal axis of the body 102.

In at least some embodiments, the length of the bridge 105 of the first arm 104 is approximately 7 to 10 millimeters and the length of the bridge 107 of the second arm 106 is approximately 5 to 8 millimeters. In at least some embodiments, the tip-to-tip distance of the extensions 104a, 104b is approximately 8 to 12 millimeters and the tip-to-tip distance of the extensions 106a, 106b is approximately 8 to 12 millimeters. In at least some embodiments, the first arm 104 forms a larger space for receiving the superior vertebra (for example, the superior spinous process) than the space formed by the second arm 106 for receiving the inferior vertebra (for example, the inferior spinous process) as vertebrae and spinous processes are naturally narrower on top and wider on the bottom.

The first and second arms 104, 106 are coupled to the body 102 using the pins 114 which each pass through opposing pin holes 141 in the body and the attachment portion 142 of each of the respective first or second arm. Each of the first and second arms 104, 106 includes a wedge engagement surface 144 that is sloping. As the actuator screw 108 is rotated in one direction, the actuator screw moves distally into or within the body 102 and, consequently, pushes the wedge 110 distally. The wedge 110 pushes against the wedge engagement surfaces 144 of the first and second arms 104, 106. Because of the slope of the wedge engagement surfaces 144, the first and second arms 104, 106 rotate about the pins 114 to move from the implantation position (FIG. 2A) to the deployed position (FIGS. 1A and 2B). In at least some embodiments, rotation in the opposite direction moves the actuator screw 108 and wedge 110 proximally which allows the first and second arms 104, 106 to return to the implantation position. In at least some embodiments, application of force to the first and second arms 104, 106 may be needed to return the first and second arms to the implantation position.

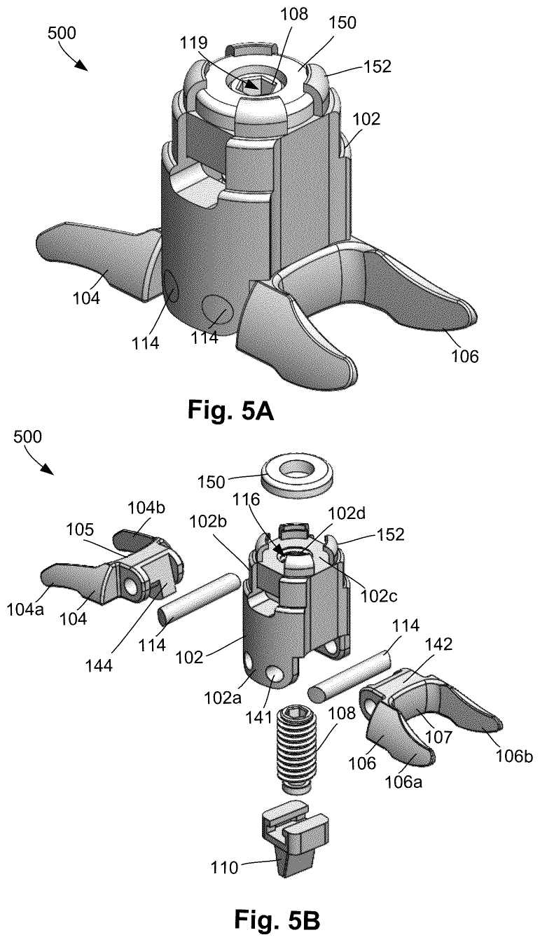

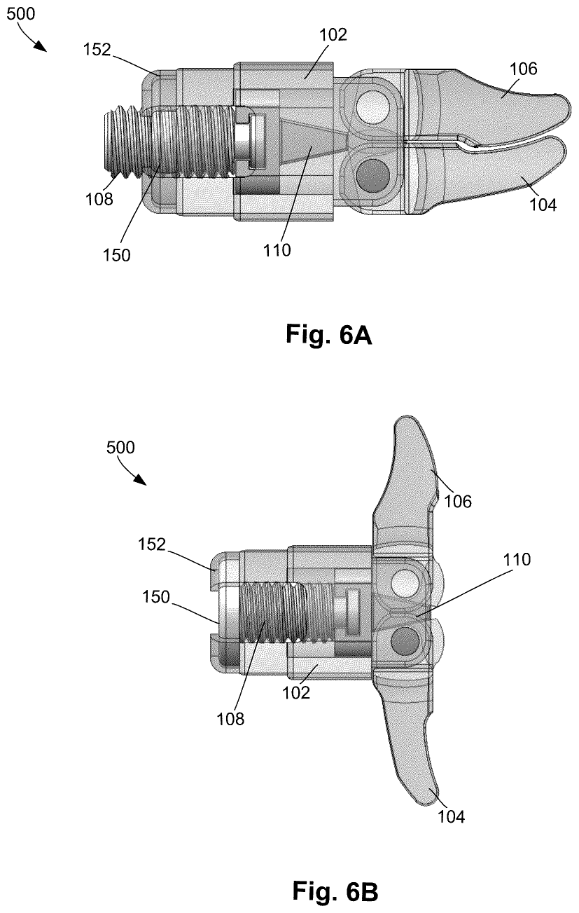

FIGS. 5A, 5B, 6A, 6B, 7A, 7B, and 8 illustrate another embodiment of an interspinous spacer 500 that includes a body 102, a first (or superior) arm 104, a second (or inferior) arm 106, an actuator screw 108, a wedge 110, a locking ring 150, and two pins 114. The components and operation of the interspinous spacer 500 are the same as the components and operation of the interspinous spacer 100 except as described herein.

The interspinous spacer 500 includes a locking ring 150, instead of the locking inserts 112 of the interspinous spacer 100, and, in at least some embodiments, the disc 120 of the actuator screw 108 does not include teeth. The actuator screw 108 is arranged so that the actuator screw engages the locking ring 150 in both the implantation position and the deployed position.

The locking ring 150 can be made of flexible material, such as polyetheretherketone (PEEK) or Nylon™ 12, that provides resistance to the rotation of the actuator screw 108, absent use of a driver tool, so that the first and second arms 104, 106 will not retract from the deployed position during use. In at least some embodiments, the material of the locking ring 150 is compressible to assist in providing the resistance.

The locking ring 150 is disposed over the proximal surface 102c of the body 102. The body 102 includes a plurality of fingers 152 that extend above the proximal surface 102c to retain the locking ring 150 and, at least in some embodiments, compress the locking ring. It will be understood that, in some embodiments, the interspinous spacer 100 can include a locking ring 150 and fingers 152 on the body in addition to the locking inserts 112. In at least some embodiments, the fingers 152 are bent after installation of the locking ring 150.

U.S. Pat. Nos. 8,123,782; 8,128,662; 8,273,108; 8,277,488; 8,292,922; 8,425,559; 8,613,747; 8,864,828; 8,945,183; 9,119,680; 9,155,572; 9,161,783; 9,393,055; 9,532,812; 9,572,603; 9,861,398; 9,956,011; 10,080,587; 10,166,047; 10,610,267; 10,653,456; 10,835,295; 10,835,297; 11,013,539; and 11,229,461, all of which are incorporated herein by reference, illustrate a variety of tools for insertion and deployment of a spacer between adjacent spinous processes. These tools can be used or modified for insertion and deployment of the spacer 100 described above.

As an example, FIGS. 9 and 10 illustrate a spacer insertion instrument 790 and a driver tool 880, respectively. The spacer insertion instrument 790 includes a cannula 791 connected to a handle 792. The spacer insertion instrument 790 defines a central passageway 793 through the handle 792 and cannula 791. The driver tool 880 is removably insertable into the central passageway 793.

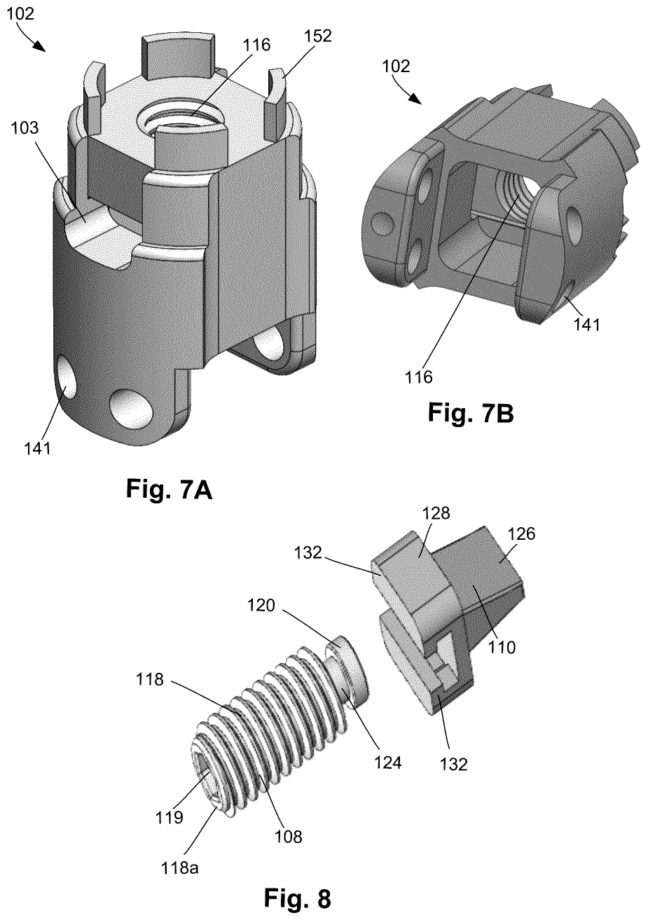

The cannula 791 includes clamps (for example, prongs) 795 to releasably clamp to the body 102 of the spacer 100, 500 (for example, to the undercut notches 103 formed on opposite sides of the body 102) for delivery of the spacer into the patient using the spacer insertion instrument 790. In at least some embodiments, the clamps 795 include extensions 796 that extend inwardly toward each other to form hooks. In at least some embodiments, the extensions 796 can engage the undercut notches 103 (FIGS. 3A and 7A) formed on opposite sides of the body 102 of the spacer 100, 500 to grip the spacer.

The cannula 791 also includes an inner shaft 797 (to which the clamps 795 are attached), an outer shaft 794, and a control 798. In at least some embodiments, the inner shaft 797 is connected to the handle 792 and the outer shaft 794 is passed over the inner shaft 797.

The outer shaft 794 translates with respect to the inner shaft 797 (or, alternatively, the inner shaft translates with respect to the outer shaft) using the control 798. The translation of the outer shaft 794 (or the inner shaft 797) operates the clamps 795. When the outer shaft 794 moves away from the clamps 795, the clamps separate to allow loading (or unloading) of the spacer 100, 500 on the spacer insertion instrument 790. When the outer shaft 794 moves toward the clamps 795, the clamps are moved together to grip the spacer 100, 500. For example, the clamps 795 can grip the undercut notches 103 formed on opposite sides of the body 102 of the spacer 100, 500. In this manner, the spacer insertion instrument 790 can hold the spacer 100, 500 for delivery of the spacer into position between adjacent spinous processes within the patient.

Turning to Figure S, a driver tool 880 includes a handle 882 at the proximal end and a spacer engaging bit 884 (for example, a socket key or hexagonal tip) at the distal end. The handle 882 and spacer engaging bit 884 are connected by a shaft 886. The driver tool 880 is sized to be inserted into the central passageway 793 of the spacer insertion instrument 790 such that the spacer engaging bit 884 at the distal end operatively connects with a spacer 100. 500 gripped by the clamps 795 of the spacer insertion instrument 790. The spacer engaging bit 884 includes features for engaging with the shaped cavity 119 (see, for example, FIG. 4) in the actuator screw 108 of the spacer 100, 500. In at least some embodiments, the driver tool 880 has a spacer engaging bit 884 that is complementary to the shaped cavity 119 in the actuator screw 108 of the spacer 100, 500. For example, the bit 884 can have a flat (like a regular screwdriver), cross (like a Phillips screwdriver), square, pentagonal, hexagonal, or octagonal shape (or any other suitable shape) with the shaped cavity 119 having a complementary shape. Rotating the driver tool 880 when engaged with the actuator screw 108 of the spacer 100, 500 rotates the actuator screw 108 to deploy the arms 104. 106 of the spacer (or. in at least some embodiments, return the arms to the implantation position).

In at least some embodiments, a small midline or lateral-to-midline incision is made in the patient for percutaneous delivery of the spacer 100, 500. In at least some embodiments, the supraspinous ligament is avoided. In at least some embodiments, the supraspinous ligament is split longitudinally along the direction of the tissue fibers to create an opening for the instrument. In at least some embodiments, one or more dilators may be used to create or enlarge the opening.

In at least some embodiments, the spacer 100, 500, in the implantation state (see, for example, FIG. 2A), is releasably attached to the spacer insertion instrument 790 as described above. In at least some embodiments, the spacer 100, 500 is inserted into a port or cannula, if one is employed, which has been operatively positioned to form an opening to the interspinous space within a patient's back. The spacer 100, 500, attached to the spacer insertion instrument 790, is inserted into the interspinous space between the spinous processes of two adjacent vertebral bodies. In at least some embodiments, the spacer 100, 500 is advanced beyond the end of a cannula or, alternatively, the cannula is pulled proximately to uncover the spacer 100, 500 connected to the spacer insertion instrument 790. Once in position, the driver tool 880 is inserted into the spacer insertion instrument 790, if not previously inserted, to engage the actuator screw 108. The driver tool 880 is rotated to rotate the actuator screw 108. The rotating actuator screw 108 begins deployment of the arms 104, 106 of the spacer 100, 500. Rotation in one direction, for example, clockwise, for example, deploys the arms 104, 106 to the deployed position (see, for example, FIG. 1A).

Other than the implantation position or deployed position, the arms 104, 106 of the spacer may be positioned in one of many partially deployed positions or intermediary positions. In at least some, embodiments, the deployment of the arms 104, 106 can be reversed by rotating the actuator screw 108 in the opposite direction, for example, counterclockwise.

In at least some embodiments, a clinician can observe with fluoroscopy or other imaging technique the positioning of the spacer 100, 500 inside the patient and then choose to reposition the spacer 100, 500 if desired. Repositioning of the spacer may involve reversing, or partially reversing, the deployment of the arms 104, 106. The arms 104, 106 of the spacer 100, 500 may then be re-deployed into the desired location. This process can be repeated as necessary until the clinician has achieved the desired positioning of the spacer in the patient.

Following deployment of the spacer, the spacer insertion instrument 790 and driver tool 880 (and any other instrumentation, such as a cannula or dilator) is removed from the body of the patient. The spacer insertion instrument 790 can be operated as described above to release the clamps 795 from the spacer 100, 500.

The above specification provides a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. An interspinous spacer, comprising:
a body having a distal portion, a proximal portion, a proximal surface, and a channel extending longitudinally from the proximal surface toward the distal portion;
an actuator screw comprising a proximal end and a shaped cavity in the proximal end, wherein the shaped cavity is configured for receiving a bit of a driver tool having a complementary shape for rotating the actuator screw using the driver tool, wherein at least a portion of the actuator screw and the channel of the body comprise complementary threading, wherein the actuator screw is at least partially disposed in, and engages, the channel of the body;
a wedge coupled to the actuator screw, wherein, as the actuator screw is rotated using the driver tool, the actuator screw and the wedge move longitudinally relative to the body; and
a first arm and a second arm, wherein the first and second arms are rotatably coupled to the distal portion of the body and configured for rotating in response to longitudinal movement of the wedge, wherein the actuator screw, wedge, first arm, and second arm are configured, upon rotation of the actuator screw in a first direction, to rotate the first and second arms from an implantation position, in which the first and second arms extend longitudinally from the distal portion of the body, to a deployed position, in which the first and second arms are rotated from the implantation position.

2. The interspinous spacer of claim 1, wherein, in the deployed position, the first and second arms extend away from the body in a lateral direction.

3. The interspinous spacer of claim 1, wherein, in the deployed position, the first and second arms are rotated from the implantation position by at least 75 degrees.

4. The interspinous spacer of claim 1, wherein the portion of the actuator screw comprising the threading is a threaded shaft and the actuator screw further comprises a disc and a cylinder coupling the disc to the threaded shaft, wherein the cylinder has an outer diameter that is smaller than outer diameters of the disc and the threaded shaft.

5. The interspinous spacer of claim 4, wherein the wedge defines an engagement slot for receiving the disc of the actuator screw and comprises two overhang portions that extend over the engagement slot are separated by a distance that is greater than the outer diameter of the cylinder and less than the outer diameters of the disc and the threaded shaft.

6. The interspinous spacer of claim 4, wherein the disc comprises a plurality of teeth arranged around a perimeter of the disc.

7. The interspinous spacer of claim 6, further comprising at least one locking inset positioned on the wedge for engagement by the disc of the actuator screw, each of the at least one locking inset comprising at least one tooth for interaction with the teeth of the disc to limit rotation of the actuator screw absent the driver tool.

8. The interspinous spacer of claim 4, further comprising a locking ring configured for engagement with the actuator screw to limit rotation of the actuator screw absent the driver tool, wherein the body comprises a plurality of fingers extending above the proximal surface to retain the locking ring on the proximal surface of the body.

9. The interspinous spacer of claim 1, further comprising two pins, wherein the body comprises two sets of opposing pin holes in the distal portion and the first and second arm comprise at least one pin hole, wherein each of the pins couples either the first or second arm to the body and extends through one of the sets of opposing pin holes of the body and the pin hole of the corresponding one of the first arm or second arm.

10. The interspinous spacer of claim 1, wherein the body comprises opposing undercut notches configured for receiving a clamp of a spacer insertion instrument.

11. A kit, comprising:
the interspinous spacer of claim 1;
a spacer insertion instrument configured to releasably grip the interspinous spacer for implantation into a patient; and
a driver tool comprising a spacer engaging bit configured to engage the actuator screw of the interspinous spacer and rotate the actuator screw by rotation of the driver tool.

12. An interspinous spacer, comprising:
a body having a distal portion, a proximal portion, a proximal surface, and a channel extending longitudinally from the proximal surface toward the distal portion;
an actuator screw comprising a threaded shaft and a disc coupled to the threaded shaft, wherein the disc comprises a plurality of teeth arranged around a perimeter of the disc and the channel of the body comprises threading complementary to the threaded shaft, wherein the actuator screw is at least partially disposed in, and engages, the channel of the body;
a wedge coupled to the actuator screw, wherein, as the actuator screw is rotated, the actuator screw and the wedge move longitudinally relative to the body;
at least one locking inset positioned on the wedge for engagement by the disc of the actuator screw, each of the at least one locking inset comprising at least one tooth for interaction with the teeth of the disc to limit rotation of the actuator screw; and
a first arm and a second arm, wherein the first and second arms are rotatably coupled to the distal portion of the body and configured for rotating in response to longitudinal movement of the wedge, wherein the actuator screw, wedge, first arm, and second arm are configured, upon rotation of the actuator screw in a first direction, to rotate the first and second arms from an implantation position, in which the first and second arms extend longitudinally from the distal portion of the body, to a deployed position, in which the first and second arms are rotated from the implantation position.

13. The interspinous spacer of claim 12, wherein, in the deployed position, the first and second arms extend away from the body in a lateral direction.

14. The interspinous spacer of claim 12, wherein the actuator screw further comprises a cylinder coupling the disc to the threaded shaft, wherein the cylinder has an outer diameter that is smaller than outer diameters of the disc and the threaded shaft, wherein the wedge defines an engagement slot for receiving the disc of the actuator screw and comprises two overhang portions that extend over the engagement slot are separated by a distance that is greater than the outer diameter of the cylinder and less than the outer diameters of the disc and the threaded shaft.

15. The interspinous spacer of claim 14, wherein the at least one locking insert is positioned within the engagement slot of the wedge.

16. A kit, comprising:
the interspinous spacer of claim 12;
a spacer insertion instrument configured to releasably grip the interspinous spacer for implantation into a patient; and
a driver tool comprising a spacer engaging bit configured to engage the actuator screw of the interspinous spacer and rotate the actuator screw by rotation of the driver tool.

17. An interspinous spacer, comprising:
a body having a distal portion, a proximal portion, a proximal surface, and a channel extending longitudinally from the proximal surface toward the distal portion;
an actuator screw comprising a threaded shaft and a disc coupled to the threaded shaft, wherein the channel of the body comprises threading complementary to the threaded shaft and the actuator screw is at least partially disposed in, and engages, the channel of the body;
a wedge coupled to the actuator screw, wherein, as the actuator screw is rotated, the actuator screw and the wedge move longitudinally relative to the body;
a locking ring configured for engagement with the actuator screw to limit rotation of the actuator screw, wherein the body comprises a plurality of fingers extending above the proximal surface to retain the locking ring on the proximal surface of the body; and
a first arm and a second arm, wherein the first and second arms are rotatably coupled to the distal portion of the body and configured for rotating in response to longitudinal movement of the wedge, wherein the actuator screw, wedge, first arm, and second arm are configured, upon rotation of the actuator screw in a first direction, to rotate the first and second arms from an implantation position, in which the first and second arms extend longitudinally from the distal portion of the body, to a deployed position, in which the first and second arms are rotated from the implantation position.

18. The interspinous spacer of claim 17, wherein the actuator screw further comprises a cylinder coupling the disc to the threaded shaft, wherein the cylinder has an outer diameter that is smaller than outer diameters of the disc and the threaded shaft, wherein the wedge defines an engagement slot for receiving the disc of the actuator screw and comprises two overhang portions that extend over the engagement slot are separated by a distance that is greater than the outer diameter of the cylinder and less than the outer diameters of the disc and the threaded shaft.

19. The interspinous spacer of claim 17, further comprising two pins, wherein the body comprises two sets of opposing pin holes in the distal portion and the first and second arm comprise at least one pin hole, wherein each of the pins couples either the first or second arm to the body and extends through one of the sets of opposing pin holes of the body and the pin hole of the corresponding one of the first arm or second arm.

20. A kit, comprising:
the interspinous spacer of claim 17;
a spacer insertion instrument configured to releasably grip the interspinous spacer for implantation into a patient; and
a driver tool comprising a spacer engaging bit configured to engage the actuator screw of the interspinous spacer and rotate the actuator screw by rotation of the driver tool.
